(19) United States
(12) Patent Application Publication
(10) Pub. No.: US 2022/0339452 A1
(43) Pub. Date: Oct. 27, 2022

(54) SYSTEM FOR DETECTING MAGNETIC RESONANCE GENERATED GRADIENT FIELD USING AN IMPLANTED MEDICAL DEVICE

(71) Applicant: Pacesetter, Inc., Sylmar, CA (US)

(72) Inventors: Shiloh Sison, Alameda, CA (US); Xi Lin Chen, Stevenson Ranch, CA (US); Xiyao Xin, Northridge, CA (US); Yash Vardhan Tiwari, Valencia, CA (US)

(21) Appl. No.: 17/670,005

(22) Filed: Feb. 11, 2022

Related U.S. Application Data
(60) Provisional application No. 63/180,141, filed on Apr. 27, 2021.

(51) Int. Cl. A61N 1/37; G01R 33/28; G01R 33/34; A61B 5/33; A61B 5/29; A61N 1/372; A61N 1/365

(52) U.S. Cl. CPC A61N 1/3718; G01R 33/287; G01R 33/34; A61B 5/33; A61B 5/29; A61N 1/37229; A61N 1/365

(57) ABSTRACT

An implantable medical device (IMD) includes electronic circuitry, and one or more processors configured to switch operation of a first coil of the electronic circuitry between the first and second modes. When in the first mode, the one or more processors are configured to manage operation of the electronic circuitry and the first coil to at least one of sense biological signals, deliver treatment for a non-physiologic condition, or wirelessly communicate with at least one of an external device or second implanted device. When in the second mode, the one or more processors are configured to manage operation of the electronic circuitry and the first coil to detect the time varying MR generated gradient field along the first axis.

### RELATED APPLICATION DATA

The present application claims priority to U.S. Ser. No. 63/180,141 Titled “SYSTEM FOR DETECTING MAGNETIC RESONANCE GENERATED GRADIENT FIELD USING AN IMPLANTED MEDICAL DEVICE” filed Apr. 27, 2021, the complete subject matter of which is expressly incorporated herein by reference in its entirety.

The present application relates to co-pending application (Ser. No. ______) Titled “METHODS AND DEVICES RELATED TO OPERATION OF AN IMPLANTABLE MEDICAL DEVICE DURING A MAGNETIC RESONANCE IMAGING” Applicant Docket No. 14255USO1; Attorney Docket 013-0422US1) filed on the same day as the present application, the complete subject matter of each of which is expressly incorporated herein by reference in its entirety.

### BACKGROUND

Embodiments herein generally relate to systems for detecting magnetic resonance generated gradient field using an active implanted medical device (IMD).

An IMD is a medical device that is configured to be implanted within a patient anatomy and commonly employs one or more leads with electrodes that either receive or deliver voltage, current or other electromagnetic pulses from or to an organ or tissue for diagnostic or therapeutic purposes. In general, IMDs include a battery, electronic circuitry, a pulse generator, a transceiver and/or a microprocessor that is configured to handle communication with an external instrument as well as control patient therapy. The components of the IMD are hermetically sealed within a metal housing.

When a patient with an active IMD undergoes a magnetic resonance (MR) scan, sensing and high voltage (HV) therapy are disabled due to the interference signals generated by the MR scanner. Interference is especially true of the signals generated by the switching gradients because the frequency content is similar to cardiac activity signals. In order to discriminate between MR generated and cardiac activity signals, accurate acquisition of the MRI generated signals is required.

One approach to maintaining sensing and HV therapy during an MRI scan is to integrate a magnetic sensor into an active IMD for the detection of a MR generated gradient field. By detecting the MR generated gradient field, the difference between the cardiac activity signals and the MR generated gradient field may be discerned. As a result, sensing and HV therapy can be maintained during an MRI scan. However, such approach adds numerous electronic circuitry into an IMD where space is limited, while adding cost to manufacturing the IMD.

### SUMMARY

In accordance with embodiments herein, an implantable medical device (IMD) is provided that includes a first coil configured to operate in first and second modes. When in the second mode, the first coil is configured to detect a first time varying MR generated gradient field along a first axis. The IMD also includes a second coil configured to detect a second time varying MR generated gradient field along a second axis, and a third coil configured to detect a third time varying MR generated gradient field along a third axis. The IMD also includes electronic circuitry, one or more processors, and a memory coupled to the one or more processors. The memory stores program instructions, and the program instructions are executable by the one or more processors to switch operation of the first coil between the first and second modes. When in the first mode, the one or more processors are configured to manage operation of the electronic circuitry and the first coil to at least one of sense biological signals, deliver treatment for a non-physiologic condition, or wirelessly communicate with at least one of an external device or second implanted device. When in the second mode, the one or more processors are configured to manage operation of the electronic circuitry and the first coil to detect the first time varying MR generated gradient field along the first axis. The one or more processors are also configured to manage operation of the second and third coils to detect the second and third time varying gradients of the magnetic field along the second and third axes, respectively.

Optionally, the biological signals include cardiac activity signals, and the program instructions are executable by the one or more processors further to detect the cardiac activity signals. The cardiac activity signals are detected utilizing the electronic circuitry in the first and second mode, while the time varying MR generated gradient field along the first axis is not detected in the first mode by the first coil. In one aspect, the second and third coils are configured to operate in the first and second modes, when in the second mode, the second and third coils configured to detect the second and third time varying gradients of the magnetic field along the second and third axes, respectively. In another aspect, the program instructions are executable by the one or more processors further to switch operation of the second and third coils between the first and second modes. When in the first mode, the one or more processors also manage operation of the electronic circuitry and the second and third coils to at least one of sense biological signals, and deliver treatment for a non-physiologic condition or wirelessly communicate with at least one of an external device or second implanted device. When in the second mode, the one or more processors manage operation of the electronic circuitry and the second and third coils to detect the second and third time varying gradients of the magnetic field along the second and third axes. In one example, the program instructions are executable by the one or more processors further to receive a manual input to switch from the first mode to the second mode, or determine the system is within a static magnetic field and switch from the first mode to the second mode responsive to detection of the static magnetic field.

Optionally, the electronic circuitry includes a transceiver, and the first coil represents a telemetry coil. When in the first mode, the telemetry coil is configured to wirelessly communicate with at least one of the external device or second implanted device. In one aspect, the first coil represents a transformer coil. When in the first mode, the transformer coil is configured to at least one of step-up or step-down a voltage between primary and secondary sides of the transformer to provide power at a select voltage to the electronic circuitry. In another aspect, the first coil represents a transformer coil coupled between a battery and a charge capacitor. When in the first mode, the transformer coil is configured to step-up a voltage level from a battery to a select voltage level to charge a capacitor. In one example, the first coil is a telemetry coil, the second coil is a transformer coil, and the third coil is an auxiliary coil. Optionally, the program instructions are executable by the one or more processors further to analyze the first time varying gradient of the magnetic field, the second time varying gradient of the magnetic field, and the third time varying MR generated gradient field to identify a signal generated by the magnetic field. The one or more processors are also configured to discriminate between the signal generated by the magnetic field and the biological signals.

In accordance with embodiments herein, a method is provided that includes under control of one or more processors monitoring biological signals, or delivering a treatment for a non-physiologic condition, with a first coil, a second coil, and a third coil in a first mode. The method also includes detecting, with the first coil, a first time varying MR generated gradient field along a first axis in a second mode, and discriminating between the biological signals and a signal generated by the magnetic field when diagnosing a medical condition based on the biological signals, or delivering the treatment.

Optionally, the method also includes under the control of the one or more processors, detecting a static magnetic field, and switching from the first mode to the second mode in response to detecting the static magnetic field. In one aspect, the electronic circuitry includes a transceiver, and the first coil represents a transceiver coil, the transceiver coil, when in the first mode, configured to wirelessly communicate with at least one of the external device or second implanted device. In another aspect, the method also includes, under the control of the one or more processors determining a period of time when the magnetic field is detectable. The method also includes switching from the first mode to the second mode during the period of time.

Optionally, discriminating between the signal generated by the magnetic field and the biological signals includes obtaining a second time varying MR generated gradient field along a second axis, obtaining the third time varying MR generated gradient field along the third axis, and determining the signal generated by the magnetic field based on the first time varying MR generated gradient field along the first axis, the second time varying MR generated gradient field along the second axis, and the third time varying MR generated gradient field along the third axis. In one embodiment, discriminating between the biological signals and a signal generated by the magnetic field also includes obtaining a candidate cardiac activity signal, and identifying the candidate cardiac activity signal as a cardiac activity signal based on the signal generated by the magnetic field. In another embodiment, identifying the candidate cardiac activity signal as a cardiac activity signal comprises comparing the candidate cardiac activity signal to the signal generated by the magnetic field. In yet another example, the method also includes under the control of the one or more processors, treating the non-physiologic condition in the second mode.

In accordance with embodiments herein an implantable medical device (IMD) is provided that includes a first coil configured to operate in first and second modes, when in the second mode, the first coil configured to detect a first time varying MR generated gradient field along a first axis. The IMD also includes a second coil configured to detect a second time varying MR generated gradient field along a second axis, and a third coil configured to detect a third time varying MR generated gradient field along a third axis. The IMD also includes electronic circuitry, one or more processors; and a memory coupled to the one or more processors. The memory stores program instructions, wherein the program instructions are executable by the one or more processors to switch operation of the first coil between the first and second modes. When in the first mode, the one or more processors also manage operation of the electronic circuitry and the first coil to at least one of sense biological signals, deliver treatment for a non-physiologic condition, or wirelessly communicate with at least one of an external device or second implanted device. When in the second mode, the one or more processors additionally manage operation of the electronic circuitry and the first coil to detect the first time varying MR generated gradient field along the first axis, and determine when to switch from the first mode to the second mode.

Optionally, to determine when to switch from the first mode to the second mode, the program instructions are executable by the one or more processors further to detect a static magnetic field. The one or more processors are also configured to switch from the first mode to the second mode in response to detecting the static magnetic field.

### DESCRIPTION OF THE DRAWINGS

FIG. 1 illustrates a schematic diagram of an IMD in accordance with embodiments herein.

FIG. 2A illustrates a schematic block diagram of an IMD in accordance with embodiments herein.

FIG. 2B illustrates a schematic block diagram of an IMD in accordance with embodiments herein.

FIG. 3 illustrates cut away view of an example IMD in accordance with embodiments herein.

FIG. 4 illustrates a perspective view of a telemetry coil in accordance with embodiments herein.

FIG. 5 illustrates a perspective view of a transformer coil in accordance with embodiments herein.

FIG. 6 illustrates a block flow diagram of a method of discriminating between a signal generated by a magnetic resonance generated gradient field and cardiac activity signals, in accordance with embodiments herein.

### DETAILED DESCRIPTION

It will be readily understood that the components of the embodiments as generally described and illustrated in the figures herein, may be arranged and designed in a wide variety of different configurations in addition to the described example embodiments. Thus, the following more detailed description of the example embodiments, as represented in the figures, is not intended to limit the scope of the embodiments, as claimed, but is merely representative of example embodiments.

Reference throughout this specification to “one embodiment” or “an embodiment” (or the like) means that a particular feature, structure, or characteristic described in connection with the embodiment is included in at least one embodiment. Thus, appearances of the phrases “in one embodiment” or “in an embodiment” or the like in various places throughout this specification are not necessarily all referring to the same embodiment.

Furthermore, the described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, etc. In other instances, well-known structures, materials, or operations are not shown or described in detail to avoid obfuscation. The following description is intended only by way of example, and simply illustrates certain example embodiments.

The methods described herein may employ structures or aspects of various embodiments (e.g., systems and/or methods) discussed herein. In various embodiments, certain operations may be omitted or added, certain operations may be combined, certain operations may be performed simultaneously, certain operations may be performed concurrently, certain operations may be split into multiple operations, certain operations may be performed in a different order, or certain operations or series of operations may be re-performed in an iterative fashion. It should be noted that, other methods may be used, in accordance with an embodiment herein. Further, wherein indicated, the methods may be fully or partially implemented by one or more processors of one or more devices or systems. While the operations of some methods may be described as performed by the processor(s) of one device, additionally, some or all of such operations may be performed by the processor(s) of another device described herein.

It should be clearly understood that the various arrangements and processes broadly described and illustrated with respect to the Figures, and/or one or more individual components or elements of such arrangements and/or one or more process operations associated of such processes, can be employed independently from or together with one or more other components, elements and/or process operations described and illustrated herein. Accordingly, while various arrangements and processes are broadly contemplated, described and illustrated herein, it should be understood that they are provided merely in illustrative and non-restrictive fashion, and furthermore can be regarded as but mere examples of possible working environments in which one or more arrangements or processes may function or operate.

All references, including publications, patent applications and patents, cited herein are hereby incorporated by reference to the same extent as if each reference were individually and specifically indicated to be incorporated by reference and were set forth in its entirety herein.

### Terms

The term “electronic circuitry” refers to any and all electronic devices, parts, traces, etc. used to vary current, voltage, resistance, charge, power, or the like using electricity. Example electronic circuitry includes capacitors, transistors, rectifiers, diodes, transformers, coils, telemetry coils, transformer coils, auxiliary coils, batteries, resistors, switches, or the like.

The terms “cardiac activity signal”, “cardiac activity signals”, “CA signal” and “CA signals” (collectively “CA signals”) are used interchangeably throughout to refer to an analog or digital electrical signal recorded by two or more electrodes positioned subcutaneous, cutaneous, or intracardiac on distal end of transvenous lead or device (leadless pacemaker), etc. where the electrical signals are indicative of cardiac electrical activity. The cardiac activity may be normal/healthy or abnormal/arrhythmic. Non-limiting examples of CA signals include ECG signals collected by cutaneous electrodes, and EGM signals collected by subcutaneous electrodes.

The term “biological signal” shall include signals measured by an IMD within the patient, where the signals are indicative of a cardiac activity characteristic, hemodynamic characteristic and/or body generated analyte. The biological signals are conveyed from the IMD within one or both IMD data sets and BGA data sets. Nonlimiting examples of biological signals include cardiac activity signals, cardiac impedance, pulmonary impedance, transthoracic impedances, accelerometer signatures, heart sounds, pulmonary arterial pressure signals, blood pressure, MCS rpm levels, MCS flow rates and the like.

The term “cardiac treatment” refers to any and all actions undertaken to treat a medical condition of the heart. Cardiac treatment includes shocks, LV shocks, MV shocks, HV shocks, pacing, HF pacing, the delivery of medicine, cardiac resynchronization therapy, or the like, including from an implantable cardioverter defibrillator (ICD).

The terms “physiologic condition” and “non-physiologic condition” are used to distinguish between a normal/healthy state of a patient condition of interest and an abnormal/unhealthy state of the patient condition of interest. Non-limiting examples of patient conditions of interest include electrical or hemodynamic cardiac behavior (e.g. normal sinus rhythms, arrhythmias, unstable hemodynamic performance), neurological behavior (e.g. pain, tremors, Parkinson's disease, tinnitus, Alzheimer's disease, and other neurological disorders measurable and treatable within the spine, brain and peripheral muscles), blood pressure, pulse oximetry levels, diabetes and other medical conditions due to imbalances/deficiencies of body generated analytes.

The term “mode” refers to a state of operation of a system, assembly, device, etc. In an example, a first mode of a system is provided when electronic circuitry functions in a first manner, and a second mode is provided when the electronic circuitry operates in a second manner. A first mode and a second mode can have similar functionalities, though some function is different. For example, a telemetry coil in a first mode can function to send IEGM cardiac signals and wirelessly communicates with at least one of an external device or second implanted device and detects a time varying MR generated gradient field along an axis. In a second mode, the telemetry coil may only send IEGM cardiac signals and wirelessly communicates with at least one of an external device or second implanted device. When used herein, time varying MR generated gradient field include, but is not limited to gradient fields and derivatives of gradient fields. In each example, a first mode and second mode are provided.

The term “discriminate”, “discriminated”, or “discriminating” refer to the act of distinguishing through determination the identification of two different items. For example, in the context of two signals, the act is to identify the first signal and the second signal and determining that the first signal and second signal are different. In another example, when a first signal presents a cardiac activity signal, a second signal presents a signal generated by a magnetic resonance generated gradient field, the first and second signals are discriminated when the cardiac activity signal is identified, and the MR generated gradient field is identified and a determination is made that two separate signals are provided. Such determination is made in one embodiment by comparing a signal from the MR generated gradient field to the cardiac activity signal.

The term “medical condition” refers to any and all disease, illness, injury, or the like related to a patient's health, organs, metal state, or the like. Medical conditions related to a patient's heart can include ventricular fibrillation (VF), arrhythmia, stroke, heart failure, heart attack, irregular heartbeat, rapid heart rate, high blood pressure, congenital heart disease, heart valve disease, atherosclerosis, pericarditis, etc.

The terms “medium-voltage shock” and “MV shock” refer to defibrillation stimulus delivered at an energy level sufficient to terminate a defibrillation episode in a heart, wherein the energy level is defined in Joules, pulse width, and/or maximum charge voltage. A MV shock from an IMD with a transvenous lead will have a different maximum energy and/or charge voltage than an MV shock from a subcutaneous IMD with a subcutaneous lead. In connection with an IMD having a transvenous lead, the terms medium voltage shock and MV shock refer to defibrillation stimulation that has an energy level that is no more than 25 J, and more preferably 15 J-25 J and/or has a maximum voltage of no more than 500V, preferably between 100-475V and more preferably between 400V-475V.

The terms “low-voltage pulse train”, “LV pulse train”, “low voltage shock”, “low voltage stimulation”, “LV shock” and the like, refer to stimulus delivered at an energy level below an MV shock energy level, and above a pacing pulse energy level, wherein the energy level is defined in Joules, maximum charge voltage and/or pulse width. In one example, the low-voltage shock train generates shocking pulses of up to 0.5 joules.

The terms “high-voltage shock” and “HV shock” refer to defibrillation stimulus delivered at an energy level sufficient to terminate a defibrillation episode in a heart, wherein the energy level is defined in Joules to be more than 25 J and/or the energy level is defined in terms of voltage to be 750V or more.

The terms “high frequency” and “HF”, as used in connection with pacing pulses and pacing therapy refer to delivering pacing pulses at a rate greater than a rate associated with anti-tachycardia pacing, namely at a rate of at least 30 Hz.

The terms “processor,” “a processor”, “one or more processors” and “the processor” shall mean one or more processors. The one or more processors may be implemented by one, or by a combination of more than one implantable medical device, a wearable device, a local device, a remote device, a server computing device, a network of server computing devices and the like. The one or more processors may be implemented at a common location or at distributed locations. The one or more processors may implement the various operations described herein in a serial or parallel manner, in a shared-resource configuration and the like.

The term “obtains” and “obtaining”, as used in connection with data, signals, information, and the like, include at least one of i) accessing memory of an external device or remote server where the data, signals, information, etc. are stored, ii) receiving the data, signals, information, etc. over a wireless communications link between the IMD and a local external device, and/or iii) receiving the data, signals, information, etc. at a remote server over a network connection. The obtaining operation, when from the perspective of an IMD, may include sensing new signals in real time, and/or accessing memory to read stored data, signals, information, etc. from memory within the IMD. The obtaining operation, when from the perspective of a local external device, includes receiving the data, signals, information, etc. at a transceiver of the local external device where the data, signals, information, etc. are transmitted from an IMD and/or a remote server. The obtaining operation may be from the perspective of a remote server, such as when receiving the data, signals, information, etc. at a network interface from a local external device and/or directly from an IMD. The remote server may also obtain the data, signals, information, etc. from local memory and/or from other memory, such as within a cloud storage environment and/or from the memory of a workstation or clinician external programmer.

Embodiments may be implemented in connection with one or more implantable medical devices (IMDs). Non-limiting examples of IMDs include one or more of neurostimulator devices, implantable cardiac monitoring and/or therapy devices. For example, the IMD may represent a cardiac monitoring device, pacemaker, cardioverter, cardiac rhythm management device, ICD, neurostimulator, leadless monitoring device, leadless pacemaker, an external shocking device (e.g., an external wearable defibrillator), and the like. For example, the IMD may be a subcutaneous IMD that includes one or more structural and/or functional aspects of the device(s) described in U.S. application Ser. No. 15/973,195, titled “Subcutaneous Implantation Medical Device With Multiple Parasternal-Anterior Electrodes” and filed May 7, 2018; U.S. application Ser. No. 15/973,219, titled “Implantable Medical Systems And Methods Including Pulse Generators And Leads” filed May 7, 2018; U.S. application Ser. No. 15/973,249, titled “Single Site Implantation Methods For Medical Devices Having Multiple Leads”, filed May 7, 2018, which are hereby incorporated by reference in their entireties. Additionally or alternatively, the IMD may include one or more structural and/or functional aspects of the device(s) described in U.S. Pat. No. 9,333,351 “Neurostimulation Method and System to Treat Apnea” and U.S. Pat. No. 9,044,710 “System and Methods for Providing A Distributed Virtual Stimulation Cathode for Use with an Implantable Neurostimulation System”, which are hereby incorporated by reference. Further, one or more combinations of IMDs may be utilized from the above incorporated patents and applications in accordance with embodiments herein.

Additionally or alternatively, the IMD may include one or more structural and/or functional aspects of the device(s) described in U.S. Pat. No. 9,216,285 “Leadless Implantable Medical Device Having Removable and Fixed Components” and U.S. Pat. No. 8,831,747 “Leadless Neurostimulation Device and Method Including the Same”, which are hereby incorporated by reference. Additionally or alternatively, the IMD may include one or more structural and/or functional aspects of the device(s) described in U.S. Pat. No. 8,391,980 “Method and System for Identifying a Potential Lead Failure in an Implantable Medical Device”, U.S. Pat. No. 9,232,485 “System and Method for Selectively Communicating with an Implantable Medical Device”, EP Application No. 0070404 “Defibrillator” and, U.S. Pat. No. 5,334,045 “Universal Cable Connector for Temporarily Connecting Implantable Leads and Implantable Medical Devices with a Non-Implantable System Analyzer”, U.S. patent application Ser. No. 15/973,126, titled “Method And System For Second Pass Confirmation Of Detected Cardiac Arrhythmic Patterns”; U.S. patent application Ser. No. 15/973,351, Titled “Method And System To Detect R-Waves In Cardiac Arrhythmic Patterns”; U.S. patent application Ser. No. 15/973,307, titled “Method And System To Detect Post Ventricular Contractions In Cardiac Arrhythmic Patterns”; and U.S. patent application Ser. No. 16/399,813, titled “Method And System To Detect Noise In Cardiac Arrhythmic Patterns” which are hereby incorporated by reference.

Additionally or alternatively, the IMD may be a leadless cardiac monitor (ICM) that includes one or more structural and/or functional aspects of the device(s) described in U.S. patent application Ser. No. 15/084,373, filed Mar. 29, 2016, entitled, “Method and System to Discriminate Rhythm Patterns in Cardiac Activity”; U.S. patent application Ser. No. 15/973,126, titled “Method And System For Second Pass Confirmation Of Detected Cardiac Arrhythmic Patterns”; U.S. patent application Ser. No. 15/973,351, titled “Method And System To Detect R-Waves In Cardiac Arrhythmic Patterns”; U.S. patent application Ser. No. 15/973,307, titled “Method And System To Detect Post Ventricular Contractions In Cardiac Arrhythmic Patterns”; and U.S. patent application Ser. No. 16/399,813, titled “Method And System To Detect Noise In Cardiac Arrhythmic Patterns”, which are expressly incorporated herein by reference.

Provided is an IMD that is able to discriminate between noise signals generated by the switching gradients generated during MRI and biological signals, including cardiac activity signals. Specifically, the gradient detection is made by three coil winding structures that are provided in the IMD, and each function to provide a function for the IMD in addition to detecting an MR generated gradient field. For example, the three coil winding structures can include telemetry coils, inductors, toroid windings, transformer coils, etc. of the IMD that can also be utilized to detect the MR generated gradient field during an MRI scan. In particular, the three coil windings selected for detecting the time varying gradient are each positioned within the IMD to have cross sections perpendicular to each other. In this manner, the time varying gradient is detected for the X, Y, and Z axis through each of the coil winding during the MRI scan for processing. In addition, the IMD can include one or more processors that continuously determines if a patient is undergoing an MRI scan to only enable the MR generated gradient field detection functionality of the three coils during an MRI scan. As such, battery life remains preserved, and non-gradient detecting functionality enabled when an MRI scan is not occurring.

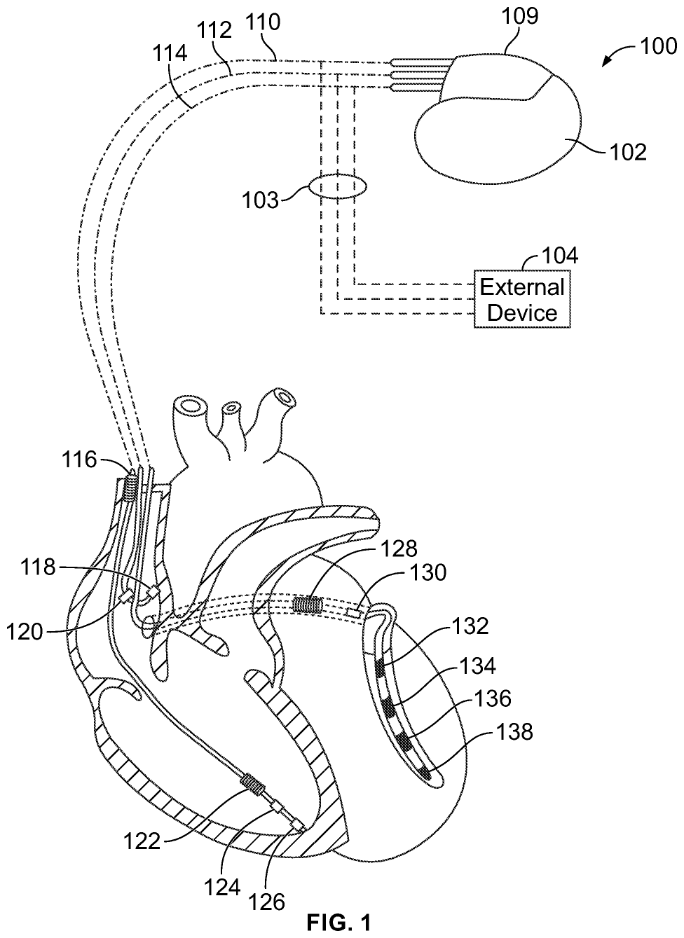

FIG. 1 illustrates an IMD 100 that in one example is a dual-chamber stimulation device capable of treating both fast and slow arrhythmias with stimulation therapy, including cardioversion, defibrillation, anti-tachycardia pacing, HF pacing, and pacing stimulation, as well as capable of detecting heart failure, evaluating its severity, tracking the progression thereof, and controlling the delivery of therapy and warnings in response thereto. The IMD 100 may be controlled to sense atrial and ventricular waveforms of interest, discriminate between two or more ventricular waveforms of interest, deliver stimulus pulses or shocks, including LV shocks, MV shocks, and HV shocks, and inhibit application of a stimulation pulse to a heart based on the discrimination between the waveforms of interest and the like. Exemplary structures for the IMD 100 are discussed and illustrated in the drawings herewith.

The IMD 100 includes a housing 102 that is joined to a header assembly 109 that holds receptacle connectors connected to a right ventricular lead 110, a right atrial lead 112, and a coronary sinus lead 114, respectively. The leads 112, 114 and 110 measure cardiac signals of the heart. While the present embodiment is described in connection with CA signals and heart conditions, it is understood that embodiments may be implemented in connection with other types of biological signals and other types of physiologic and non-physiologic conditions. The right atrial lead 112 includes an atrial tip electrode 118 and an atrial ring electrode 120. The coronary sinus lead 114 includes a left atrial ring electrode 128, a left atrial coil electrode 130 and one or more left ventricular electrodes 132-138 (e.g., also referred to as P1, M1, M2 and D1) to form a multi-pole LV electrode combination. The right ventricular lead 110 includes an RV tip electrode 126, an RV ring electrode 124, an RV coil electrode 122, and an SVC coil electrode 116. The leads 112, 114 and 110 detect IEGM signals that are processed and analyzed as described herein. The leads 112, 114 and 110 also delivery therapies as described herein.

During implantation, an external device 104 is connected to one or more of the leads 112, 114 and 110 through temporary inputs 103. The inputs 103 of the external device 104 receive IEGM signals from the leads 112, 114 and 110 during implantation and display the IEGM signals to the physician on a display. Hence, the external device 104 receives the IEGM cardiac signals through telemetry circuit inputs. The physician or another user controls operation of the external device 104 through a user interface. While the example embodiment of FIG. 1 illustrates an IMD 100 that includes leads, such embodiment is for exemplary purposes only, in other example embodiments, the IMD may be a leadless IMD.

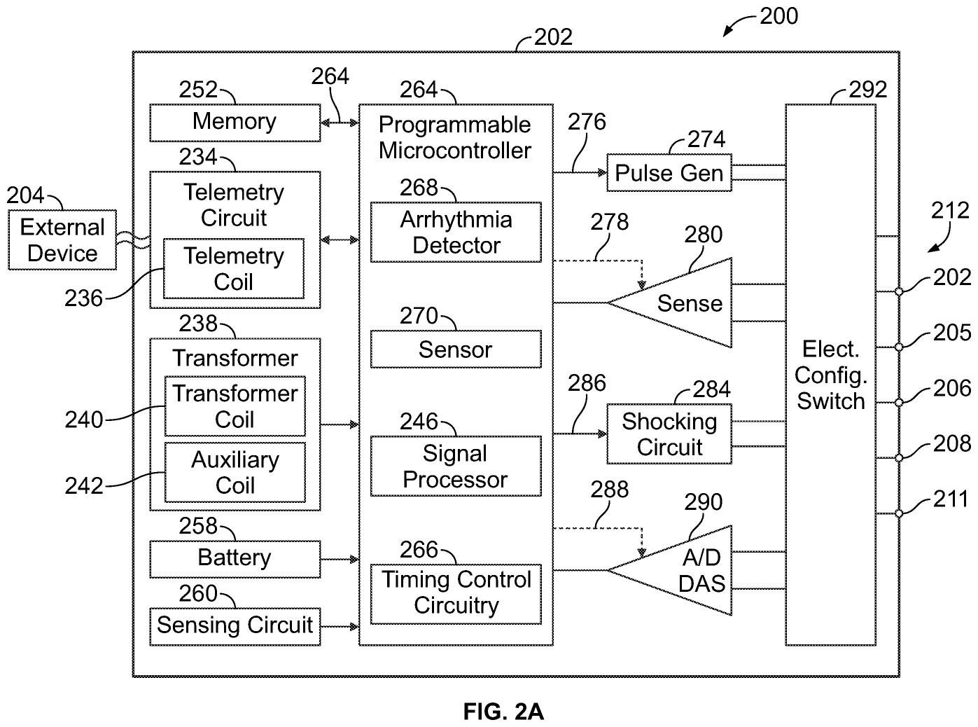

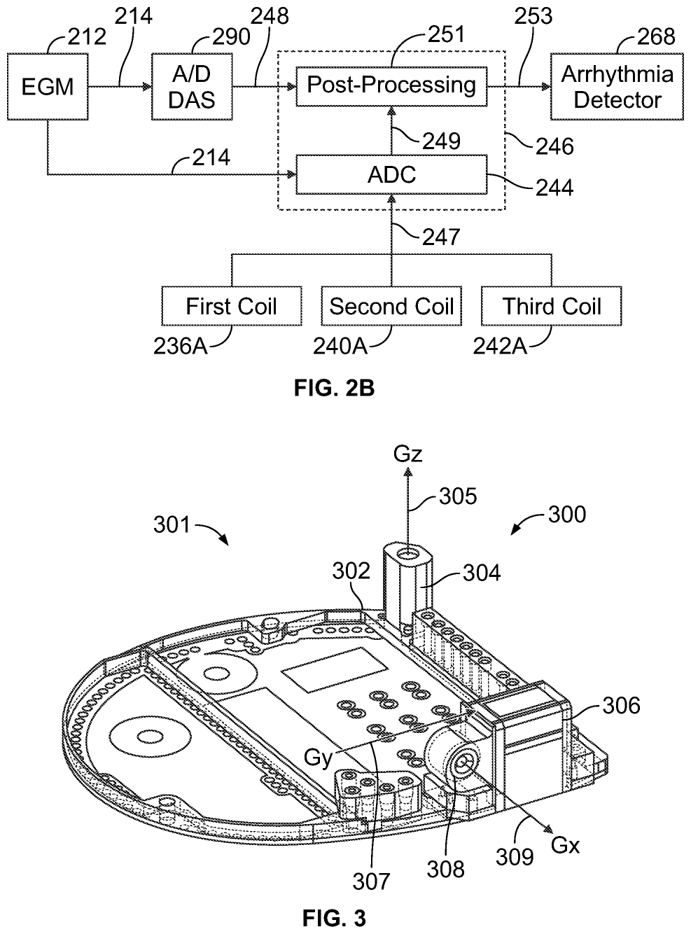

FIG. 2A illustrates and example block diagram of a IMD 200 that is implanted into the patient as part of the implantable cardiac system. While described as an implantable cardiac system, this is for exemplary purposes only, and other IMDs are contemplated. In this embodiment, the IMD 200 may be implemented as a full-function biventricular pacemaker, equipped with both atrial and ventricular sensing and pacing circuitry for four chamber sensing and stimulation therapy (including both pacing and shock treatment). The pacing circuitry in one example includes HF pacing. Optionally, the IMD 200 may provide full-function cardiac resynchronization therapy. Alternatively, the IMD 200 may be implemented with a reduced set of functions and components. For instance, the monitoring device may be implemented without ventricular sensing and pacing.

The IMD 200 has a housing 201 to hold the electronic/computing components. The housing 201 (which is often referred to as the “can”, “case”, “encasing”, or “case electrode”) may be programmably selected to act as the return electrode for certain stimulus modes. Housing 201 further includes a connector (not shown) with a plurality of terminals 202, 205, 206, 208, and 211 for electrodes 212. The type and location of each electrode may vary. For example, the electrodes may include various combinations of ring, tip, coil and shocking electrodes and the like.

The IMD 200 also includes a telemetry circuit 234 that as a primary function allows intracardiac electrograms and status information relating to the operation of the IMD 200 (as contained in the microcontroller 264 or memory 252) to be sent to the external device 204 through the established communication link 250. Specifically, the telemetry circuit wirelessly communicates with at least one of the external device 204 or second implanted device. In addition, the telemetry circuit 234 includes a telemetry coil 236 that may be positioned to detect or obtain a time varying MR generated gradient field along an axis when a patient is undergoing an MRI scan as a secondary function, in addition to communicating the electrograms and status information relating to the operation of the IMD 200. In one example, the telemetry coil 236 is utilized to detect or obtain the time varying MR generated gradient field by using methodologies as described in relation to U.S. Pat. No. 8,965,526 to Zhang et al. filed Oct. 10, 2012, that is incorporated by reference in full herein. In this manner, the telemetry coil 236 performs dual functions within the IMD 200.

The IMD 200 may also include a transformer 238 that includes a transformer coil 240, and an auxiliary coil 242. The transformer 238 as a primary function is configured to at least one of step-up or step-down a voltage between primary and secondary sides of the transformer to provide power at a select voltage to the electronic circuitry. The transformer coil 240, and auxiliary coil 242 may also be positioned to provide a secondary function of detecting a time varying MR generated gradient field along an axis when a patient is undergoing an MRI. Specifically, when each of the telemetry coil 236, transformer coil 240, and auxiliary coil 242 are positioned at 90° to one another, each axis of a static magnetic field formed by an MRI may be detected and communicated to a microcontroller 264 for analysis to detect the gradient change of the magnetic field. As such, the microcontroller 264 may distinguish between the detected gradient change in the magnetic field, and cardiac activity signals.

FIG. 2B illustrates an example block flow diagram of how both biological signals, that in this example are candidate cardiac activity (CA) signals, and signals of a time varying gradient from a first coil 236A, second coil 240A, and third coil 242A are processed. Specifically, while the present embodiment is described in connection with CA signals and heart conditions, it is understood that embodiments may be implemented in connection with other types of biological signals and other types of physiologic and non-physiologic conditions. In one example the first coil 236A is a telemetry coil, the second coil 240A is a transformer coil, and the third coil 242A is an auxiliary coil.

In the example, electrodes 212 obtain candidate cardiac activity signals 214 in analog form. In one example, the candidate cardiac activity signal is an EGM. The analog candidate cardiac activity signal can then either be passed to an analog-to-digital (A/D) data acquisition system (DAS) 290 coupled to one or more electrodes via a switch 292 to sample candidate cardiac activity signals across any pair of desired electrodes, or to an A/D converter 244 of a signal processor 246 that receives input 247 from each of the telemetry coil 236, transformer coil 240, and auxiliary coil 242 when functioning to detect the time varying MR generated gradient field along an axis.

In each instance, the A/D device (e.g. A/D DAS 290, or A/D converter 244) then provides a digitized signal 248, 249 to a post processor 251 of the signal processor 246. The signal processor 246 then identifies signals that are generated as a result of a magnetic resonance generated gradient field based on the input 247 of the first coil 236A, second coil 240, and third coil 242 to make a determination regarding whether the candidate cardiac activity signals from the electrodes 212 are resulting from the magnetic resonance generated gradient field. In one example, the digitized input from coils is compared to the digitize input from the electrodes. Based on the determination, the signal processor discards candidate cardiac activity signals that are merely signals based on the magnetic resonance generated gradient field, while identifying the cardiac activity signals 253 for diagnosis, treatment, or the like. As an example, the cardiac activity signal 253 can be provided to the arrhythmia detector 268 for additional analysis.

With reference back to FIG. 2A, the programmable microcontroller 264 controls various operations of the IMD 200. Microcontroller 264 includes a microprocessor (or equivalent control circuitry), RAM and/or ROM memory, logic and timing circuitry, state machine circuitry, and I/O circuitry. The IMD 200 further includes a first chamber pulse generator 274 that generates stimulation pulses for delivery by one or more electrodes coupled thereto. The pulse generator 274 is controlled by the microcontroller 264 via control signal 276. The pulse generator 274 is coupled to the select electrode(s) via an electrode configuration switch 292, which includes multiple switches for connecting the desired electrodes to the appropriate I/O circuits, thereby facilitating electrode programmability. The switch 292 is controlled by a control signal 286 from the microcontroller 264.

Microcontroller 264 is illustrated to include timing control circuitry 266 to control the timing of the stimulation pulses (e.g., pacing rate, atrio-ventricular (AV) delay, atrial interconduction (A-A) delay, or ventricular interconduction (V-V) delay, etc.). Microcontroller 264 also has an arrhythmia detector 268 for detecting arrhythmia conditions. Although not shown, the microcontroller 264 may further include other dedicated circuitry and/or firmware/software components that assist in monitoring various medical conditions, including of the patient's heart and managing pacing therapies, including HF pacing.

The microcontroller 264 is also configured to operate in different modes. For example, in one embodiment, the IMD may include electronic circuitry that has dual functionality. Such dual functionality may include a primary functionality for day to day activities of a patient, while a secondary functionality may be provided when a patient is undergoing a procedure such as an MRI scan. For example, in a first mode, the electronic circuitry can include components that sense biological signals, deliver treatment for a non-physiologic condition, wirelessly communicate with at least one of an external device or second implanted device, etc. Meanwhile, in the second mode, the same electronic components of the electronic circuitry add the functionality of detecting the time varying MR generated gradient field along an axis, in addition to sensing biological signals, delivering treatment for a non-physiologic condition, wirelessly communicating with at least one of an external device or second implanted device, etc. In this manner, the IMD may be programmable to switch from a first mode to a second mode (e.g. MRI mode) such that certain components of the electronic circuitry of the IMD function to detect the time varying MR generated gradient field along an axis during an MRI scan in addition to their first mode functionality.

The IMD 200 also includes one or more sensors 270. The one or more sensors 270 can include physiological sensors that detect characteristics associated with the heart of the patient. Alternatively, the one or more sensors 270 can be environmental sensors that detect characteristics associated with the environment of the patient. In one example, the one or more sensors detect a static magnetic field, such as one produced by an MRI scan, around the patient. In this manner, the one or more sensors may be utilized to detect information and data utilized to determine if the patient is undergoing an MRI scan. To this end, upon detection of the static magnetic field, the microcontroller can switch the IMD from a first mode to a second mode in order to detect the time varying MR generated gradient field with the electronic circuitry.

The IMD 200 is further equipped with a communication modem (modulator/demodulator) to enable wireless communication with other devices, implanted devices, and/or external devices. The IMD 200 also includes sensing circuitry 280 selectively coupled to one or more electrodes that perform sensing operations, through the switch 292, to detect the presence of cardiac activity.

The output of the sensing circuitry 280 is connected to the microcontroller 264 which, in turn, triggers or inhibits the pulse generator 274 in response to the absence or presence of cardiac activity. The sensing circuitry 280 receives a control signal 278 from the microcontroller 264 for purposes of controlling the gain, threshold, polarization charge removal circuitry (not shown), and the timing of any blocking circuitry (not shown) coupled to the inputs of the sensing circuitry.

In the example of FIG. 2A, a single sensing circuit 280 is illustrated. Optionally, the IMD 200 may include multiple sensing circuit, similar to sensing circuit 280, where each sensing circuit is coupled to one or more electrodes and controlled by the microcontroller 264 to sense electrical activity detected at the corresponding one or more electrodes. The sensing circuit 280 may operate in a unipolar sensing configuration or in a bipolar sensing configuration.

The microcontroller 264 is also coupled to a memory 252 by a suitable data/address bus 262. The programmable operating parameters used by the microcontroller 264 are stored in memory 252 and used to customize the operation of the IMD 200 to suit the needs of a particular patient. Such operating parameters define, for example, pacing pulse amplitude, pulse duration, electrode polarity, rate, sensitivity, automatic features, HF pacing features, arrhythmia detection criteria, and the amplitude, waveshape and vector of each shocking pulse to be delivered to the patient's heart within each respective tier of therapy. Such shocking pulse can be an LV shock, MV shock, HV shock, etc.

A battery 258 provides operating power to all of the components in the IMD 200. The IMD 200 further includes an impedance measuring circuit 260, which can be used for many things, including: lead impedance surveillance during the acute and chronic phases for proper lead positioning or dislodgement; detecting operable electrodes and automatically switching to an operable pair if dislodgement occurs; measuring thoracic impedance for determining shock thresholds; detecting when the device has been implanted; measuring stroke volume; and detecting the opening of heart valves; and so forth. The impedance measuring circuit 260 is coupled to the switch 292 so that any desired electrode may be used. The IMD 200 can be operated as an implantable cardioverter/defibrillator (ICD) device, which detects the occurrence of an arrhythmia and automatically applies an appropriate electrical shock therapy to the heart aimed at terminating the detected arrhythmia. To this end, the microcontroller 264 further controls a shocking circuit 284 by way of a control signal 286, including for LV shocks, MV shocks, HV shocks, etc.

FIG. 3 illustrates a cut away view of an example IMD 300. In one example, IMD 300 is the IMD of FIGS. 1-2B. In the embodiment, the casing of the IMD is removed to illustrate electronic circuitry 301 of the IMD. Specifically, the IMD 300 includes a substrate 302 that receives the electrical components 301. The electronic circuitry 301 is disposed on the substrate 300 and include a first coil 304 that is configured to detect a time varying MR generated gradient field along a Z-axis 305, second coil 306 that is configured to detect a time varying MR generated gradient field along a Y-axis 307, and third coil 308 that is configured to detect a time varying MR generated gradient field along an X-axis 309.

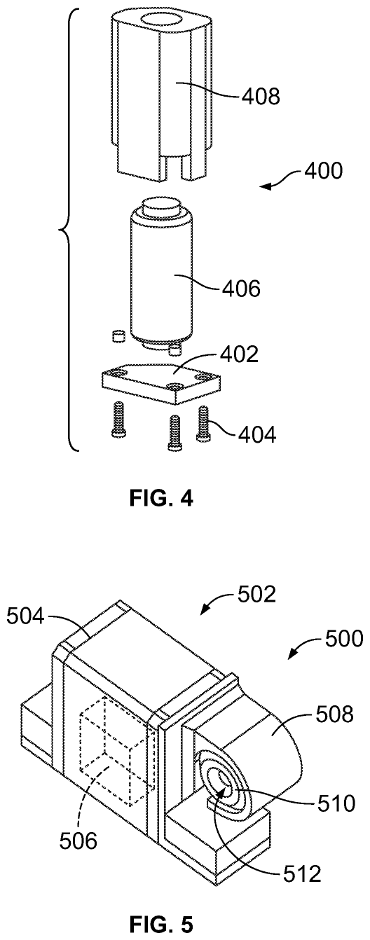

In one example embodiment, the first coil 304 may be a telemetry coil that is part of telemetry circuitry and functions to send intracardiac electrograms and status information relating to the operation of the IMD 300 to an external device. A telemetry coil 400 is illustrated in more detail in FIG. 4. As shown, the telemetry coil 400 includes a telemetry substrate 402 with pin elements 404 to provide an electrical coupling with a core and windings 406. The core and windings 406 are encased in a frame 408 for containing the core and windings 406.

With reference back to FIG. 3, in this example, the first coil 304 is positioned to detect the time varying MR generated gradient field created by an MRI scan. In this manner, when the first coil 304 is a telemetry coil, the first coil is configured to both communicate wirelessly with at least one of an external device or second implanted device, and detect a time varying gradient of a magnetic field. While in this example, the first coil 304 is positioned to detect the time varying MR generated gradient field along the Z-axis, in another example, the first coil 304 can be positioned to detect the time varying MR generated gradient field along the X-axis, or Y-axis. In yet another example, an IMD can include a telemetry coil that is not utilized to detect a time varying gradient along any axis, and instead three other coils are utilized to detect the time varying gradient each axis of a magnetic field.

In the example embodiment of FIG. 3, the second coil 306 is a transformer coil. As illustrated in FIG. 5, the electronic circuitry 301 can include a transformer 500 that has a transformer coil 502. The transformer 500 also includes an exterior shield 504 that houses a core and windings 506 of the transformer coil 502. In one example, the transformer 500 may also include a toroid base 508 that includes a toroid core with windings 510 that provide an auxiliary coil 512. In such an example, the auxiliary coil 512 may be the third coil 308. The transformer coil in one example is configured to at least one of step-up or step-down a voltage between primary and secondary sides of the transformer to provide power at a select voltage to the electronic circuitry. In another example, the auxiliary coil 512 is a transformer coil that is coupled between a battery and a charge capacitor, and is configured to step-up a voltage level from a battery to a select voltage level to charge a capacitor.

With reference back to FIG. 3, in this example, the second coil 306 is positioned to detect the time varying MR generated gradient field along the Y-axis. Specifically, the second coil 306 is at a 90° angle to the first coil 304 such that while the first coil 304 detects the time varying MR generated gradient field of the MRI along the Z-axis 305, the second coil 306 detects the time varying MR generated gradient field along the Y-axis 307. Meanwhile, the third coil 308 is positioned at a 90° angle to both the first coil 304 and the second coil 306, such that the third coil 308 detects the time varying MR generated gradient field of the MRI scan along the X-axis 309. While in one example, the first coil 304 can be a telemetry coil that detects the time varying MR generated gradient field of the MRI scan along the Z-axis 305, the second coil 306 can be a transformer coil that detects the time varying MR generated gradient field of the MRI scan along the Y-axis 307, and the third coil 308 can be an auxiliary coil that detects the time varying MR generated gradient field of the MRI scan along X-axis 309, in other embodiments, the first coil 304, second coil 306, and third coil 308 may be positioned to detect a different axis. Specifically, based on spatial considerations, the different coils 304, 306, 308 may be positioned to detect the different axes. To this end, other coils may be utilized instead of a telemetry coil, transformer coil, and auxiliary coil to detect the different axes. Still, by detecting each axis by positioning each coil at a 90° angle to one another, the time varying MR generated gradient field generated by the MRI scan can be distinguished from cardiac activity signals without the need to provide additional time varying gradient detecting components within the IMD. As a result, spatial conditions are improved, and expense decreased by providing coils within the IMD with dual functionality.

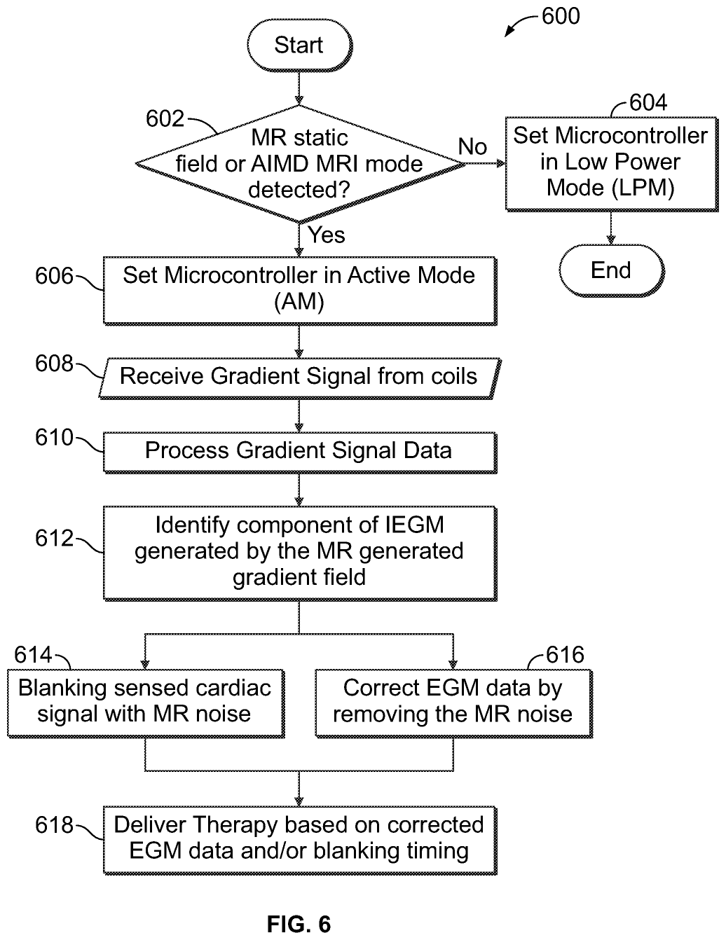

FIG. 6 illustrates a method 600 of discriminating between variances in a magnetic field created by an MRI scan and biological signals such as cardiac activity signals provided by a patient. While the present embodiment is described in connection with CA signals and heart conditions, it is understood that embodiments may be implemented in connection with other types of biological signals and other types of physiologic and non-physiologic conditions. In one example, the IMD and/or the electrical circuitry of FIGS. 1-5 are utilized to perform the process.

At 602, one or more processors determine if a patient is undergoing, or about to undergo an MRI scan. In one example, the IMD includes an environmental sensor that detects a static magnetic field generated by an MRI scan. Upon detecting the static magnetic field, the IMD switches from a first mode to a second mode, or MR mode. In particular, in one example, in the first mode components of electronic circuitry of the IMD can sense biological signals, deliver treatment for a non-physiologic condition, wirelessly communicate with at least one of an external device or second implanted device, etc. In the second mode the same electronic components of the electronic circuitry add the functionality of detecting a time varying gradient of a magnetic field. Specifically, the electronic components may be a telemetry coil, transformer coil, auxiliary coil, or the like.

Alternatively, to determine if a patient is undergoing, or about to undergo an MRI scan, the one or more processors may obtain a manual input by a patient, clinician, etc. In another example, the IMD may receive wireless communication to switch the IMD from the first mode to the second mode. In another example, a wireless communication may provide a determined time period for operating the IMD in the second mode. In yet another example, the one or more processor may obtain patient information, such as a scheduled time on a calendar on an electronic device of the patient such as a smart phone, personal digital assistant (PDA), etc. that wirelessly communicates with the IMD, including through Bluetooth. Through such communication, the scheduled time of an MRI may be provided, causing the one or more processors to automatically operate in the second mode during the time period associated with the MRI scan. The one or more processors may utilize a lookup table, decision tree, mathematical model, mathematical function, artificial intelligence algorithm, other algorithm, or the like to determine that the patient is undergoing an MRI scan, or is about to undergo an MRI scan.

In each instance, the IMD only operates in the second mode during the MRI scan to reduce the amount of time the battery of the IMD is being utilized to detect the MR. In this manner, the advantage of continuing to monitor cardiac activity signals and provide cardiac treatment during an MRI scan is realized, without reducing the life of the battery of the IMD to a point where such benefit is detrimental to the overall operation of the IMD.

If at 602, a determination is made that the IMD is not undergoing, or about to undergo an MRI scan, then at 604, the one or more processors set the microprocessor in a low power mode. Specifically, in the low power mode, the IMD does not function to detect a time varying MR generated gradient field along an axis during an MRI scan. Instead, the electrical components of the IMD only provide their primary function. In this manner, the IMD only functions to detect the time varying MR generated gradient field along an axis during an MRI, and not during times when the patient is not undergoing an MRI. As a result, the battery of the IMD is conserved.

In particular, an analysis was provided on an IMD that functioned utilizing the process provided in FIG. 6. The life of the battery of the IMD was determined to be 7.74 years based on operating conditions where the IMD did not include an MR mode. Specifically, such IMDs are not capable of distinguishing between a time varying MR generated gradient field formed as a result of an MRI and cardiac activity signals. As a result, such IMD are turned off during the MRI resulting in risk to the patient. However, when the IMD provided an MRI mode that presents an ability to distinguish between the time varying MR generated gradient field during an MRI and cardiac activity signals, the life of the battery of the IMD only decreased by 89 days. Thus, by only operating the IMD in a MR mode during an MRI scan, battery life is saved such that gaining the benefit of distinguishing between signals generated as a result of the time varying MR generated gradient field formed as a result of an MRI scan and cardiac activity signals does not reduce of life of the battery causing the technology not to be practical and usable in a real-world setting. Therefore, not only is the MR mode provide better health for a user, because existing electronic circuitry is utilized to detect the time vary gradient of the magnetic field, spatial improvements and cost reductions are also realized.

In one example, the one or more processors determine whether to operate in a first mode where the electronic circuitry of the IMD detects cardiac activity signals while not detecting a magnetic resonance generated gradient field with a first coil, second coil and third coil, or a second mode where the electronic circuitry of the IMD detects the cardiac activity signals while also detecting the magnetic resonance generated gradient field with the first coil, second coil, and third coil. In one example, the second mode is considered an MR mode.

If at 602 the one or more processors determine an MRI scan is occurring, or will occur in the near future and a second mode is needed, the one or more processors at 606 switch from the first mode to a second mode. In the second mode the first coil, second coil, and third coil detect the time varying gradient of a magnetic field. In particular, one or more coils are provided in the IMD that provide a primary functionality related to the IMD, and a secondary functionality for detecting the time varying gradient of a magnetic field. In one example, at least three coils are provided, each detecting the time varying gradient of a different axis of the magnetic field. Specifically, the one or more processors in the second mode monitor and detect the time varying MR generated gradient field along each axis of each individual coil. In one example, a telemetry coil is positioned and detects the time varying gradient along a Z-axis, a transformer coil is positioned and detects the time varying gradient along a Y-axis, and an auxiliary coil is positioned and detects the time varying gradient along an X-axis. As such, the time varying MR generated gradient field of the MRI scan can be detected. In other example embodiments, other coils within the IMD may be utilized to provide detection of the time varying gradient of the magnetic field. Similarly, the axis upon which each coil detects may vary based on spatial considerations related to the IMD.

At 608, the one or more processors receive a gradient signal from each of the individual coils. In this manner, a gradient signal from than X-axis, Y-axis, and Z-axis are all detected and obtained for analysis.

At 610, the one or more processors process gradient signal data. In particular, from the received gradient signals from each axis, gradient signal data is formed. The gradient signal data may be processed for analysis. In one example, an A/D converter is utilized to digitize the gradient signals into the gradient signal data. By digitizing the gradient signals into gradient signal data, the gradient signal data may be utilized to identify signals being generated as a result of the magnetic field, as opposed to cardiac activity signals.

At 612, the one or more processors analyze the gradient signal data to identify signals generated by the magnetic resonance generated gradient field generated as a result of the magnetic field of the MRI scan. In particular, based on the time varying MR generated gradient field as measured by the first coil, second coil, and third coil, signals generated by the magnetic resonance generated gradient field can be identified. In example embodiments, the one or more processors may utilize an algorithm, mathematical function, mathematical model, mathematical calculations, lookup table, or the like to make determinations related to whether the signals are the same, or substantially similar.

At 614, the one or more processors blank a sensed cardiac signal utilizing MR noise. Once a sensed signal is identified as a result of the magnetic resonance generated gradient field, the sensed signal is blanked, or prevented from being utilized when providing treatment. By having a system and method that detects and identifies the signals generated by the magnetic resonance generated gradient field generated as a result of the magnetic field of the MRI scan, such signals can be accounted for, allowing treatment to continue during the MRI scan.

Alternatively, at 616, the one or more processors correct the cardiac activity signal data by removing the MR noise. Again, instead of basing treatment on the MR generated signals, by correcting the CA signal data by removing the MR noise, treatment is only provided based on CA signals. In this manner, steps 614 and 616 both provide remedies to prevent false positive signals caused by an MRI scan resulting in diagnosing a heart condition, medical condition, etc. or providing a medical treatment, cardiac treatment, or the like that can be harmful and undesired to the patient.

At 618, the one or more processors delivery therapy based on either the corrected CA signal data where MR noise has been removed (step 616), or provides therapy based on the blanking timing that accounts for the MR signals identified (step 614). By providing the treatment accounting for the MR signals, false positive signals are prevented, allowing continued treatment of the patient even during an MRI scan.

In all, by repositioning already existing electronic circuitry of the IMD to detect a time varying gradient signal along the X, Y, and Z axes, an IMD is able to distinguish between time varying gradient signals resulting from magnetic resonance and the cardiac activity signals. Because existing electrical components are being utilized, and simply providing an additional detection function, electrical component hardware costs do not increase. In addition, the size of the IMD does not have to be increased to accommodate the additional electrical components. Finally, by only operating a second, MR, mode when an MRI scan is being conducted, only a minimal amount of battery is consumed such that longevity of the IMD can be maintained. As a result, reliability is improved without increasing manufacturing costs or complexities, all when only slightly detracting from the IMD life.

### Closing

It should be clearly understood that the various arrangements and processes broadly described and illustrated with respect to the Figures, and/or one or more individual components or elements of such arrangements and/or one or more process operations associated of such processes, can be employed independently from or together with one or more other components, elements and/or process operations described and illustrated herein. Accordingly, while various arrangements and processes are broadly contemplated, described and illustrated herein, it should be understood that they are provided merely in illustrative and non-restrictive fashion, and furthermore can be regarded as but mere examples of possible working environments in which one or more arrangements or processes may function or operate.

As will be appreciated by one skilled in the art, various aspects may be embodied as a system, method, or computer (device) program product. Accordingly, aspects may take the form of an entirely hardware embodiment or an embodiment including hardware and software that may all generally be referred to herein as a “circuit,” “module” or “system.” Furthermore, aspects may take the form of a computer (device) program product embodied in one or more computer (device) readable storage medium(s) having computer (device) readable program code embodied thereon.

Any combination of one or more non-signal computer (device) readable medium(s) may be utilized. The non-signal medium may be a storage medium. A storage medium may be, for example, an electronic, magnetic, optical, electromagnetic, infrared, or semiconductor system, apparatus, or device, or any suitable combination of the foregoing. More specific examples of a storage medium would include the following: a portable computer diskette, a hard disk, a random access memory (RAM), a dynamic random access memory (DRAM), a read-only memory (ROM), an erasable programmable read-only memory (EPROM or Flash memory), a portable compact disc read-only memory (CD-ROM), an optical storage device, a magnetic storage device, or any suitable combination of the foregoing.

Program code for carrying out operations may be written in any combination of one or more programming languages. The program code may execute entirely on a single device, partly on a single device, as a stand-alone software package, partly on single device and partly on another device, or entirely on the other device. In some cases, the devices may be connected through any type of network, including a local area network (LAN) or a wide area network (WAN), or the connection may be made through other devices (for example, through the Internet using an Internet Service Provider) or through a hard wire connection, such as over a USB connection. For example, a server having a first processor, a network interface, and a storage device for storing code may store the program code for carrying out the operations and provide this code through its network interface via a network to a second device having a second processor for execution of the code on the second device.

Aspects are described herein with reference to the figures, which illustrate example methods, devices, and program products according to various example embodiments. The program instructions may be provided to a processor of a general-purpose computer, special purpose computer, or other programmable data processing device or information handling device to produce a machine, such that the instructions, which execute via a processor of the device implement the functions/acts specified. The program instructions may also be stored in a device readable medium that can direct a device to function in a particular manner, such that the instructions stored in the device readable medium produce an article of manufacture including instructions which implement the function/act specified. The program instructions may also be loaded onto a device to cause a series of operational steps to be performed on the device to produce a device implemented process such that the instructions which execute on the device provide processes for implementing the functions/acts specified.

The units/modules/applications herein may include any processor-based or microprocessor-based system including systems using microcontrollers, reduced instruction set computers (RISC), application specific integrated circuits (ASICs), field-programmable gate arrays (FPGAs), logic circuits, and any other circuit or processor capable of executing the functions described herein. Additionally, or alternatively, the modules/controllers herein may represent circuit modules that may be implemented as hardware with associated instructions (for example, software stored on a tangible and non-transitory computer readable storage medium, such as a computer hard drive, ROM, RAM, or the like) that perform the operations described herein. The above examples are exemplary only, and are thus not intended to limit in any way the definition and/or meaning of the term “controller.” The units/modules/applications herein may execute a set of instructions that are stored in one or more storage elements, in order to process data. The storage elements may also store data or other information as desired or needed. The storage element may be in the form of an information source or a physical memory element within the modules/controllers herein. The set of instructions may include various commands that instruct the modules/applications herein to perform specific operations such as the methods and processes of the various embodiments of the subject matter described herein. The set of instructions may be in the form of a software program. The software may be in various forms such as system software or application software. Further, the software may be in the form of a collection of separate programs or modules, a program module within a larger program or a portion of a program module. The software also may include modular programming in the form of object-oriented programming. The processing of input data by the processing machine may be in response to user commands, or in response to results of previous processing, or in response to a request made by another processing machine.

It is to be understood that the subject matter described herein is not limited in its application to the details of construction and the arrangement of components set forth in the description herein or illustrated in the drawings hereof. The subject matter described herein is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

It is to be understood that the above description is intended to be illustrative, and not restrictive. For example, the above-described embodiments (and/or aspects thereof) may be used in combination with each other. In addition, many modifications may be made to adapt a particular situation or material to the teachings herein without departing from its scope. While the dimensions, types of materials and coatings described herein are intended to define various parameters, they are by no means limiting and are illustrative in nature. Many other embodiments will be apparent to those of skill in the art upon reviewing the above description. The scope of the embodiments should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects or order of execution on their acts.

What is claimed is:

1. An implantable medical device (IMD), comprising:
a first coil configured to operate in first and second modes, when in the second mode, the first coil configured to detect a time varying MR generated gradient field along a first axis;
a second coil configured to detect a time varying MR generated gradient field along a second axis;
a third coil configured to detect a time varying MR generated gradient field along a third axis;
electronic circuitry;
one or more processors; and
a memory coupled to the one or more processors, wherein the memory stores program instructions, wherein the program instructions are executable by the one or more processors to:
switch operation of the first coil between the first and second modes;
when in the first mode, manage operation of the electronic circuitry and the first coil to at least one of sense biological signals, deliver treatment for a non-physiologic condition or wirelessly communicate with at least one of an external device or second implanted device;
when in the second mode, manage operation of the electronic circuitry and the first coil to detect the time varying MR generated gradient field along the first axis; and
manage operation of the second and third coils to detect the time varying gradients of the magnetic field along the second and third axes, respectively.

2. The IMD of claim 1, wherein the biological signals include cardiac activity signals, the program instructions are executable by the one or more processors further to:
detect the cardiac activity signals while not detecting the first time varying MR generated gradient field along the first axis in the first mode.

3. The IMD of claim 1, wherein the second and third coils are configured to operate in the first and second modes, when in the second mode, the second and third coils configured to detect the time varying MR generated gradient field along the second and third axes, respectively.

4. The IMD of claim 3, wherein the program instructions are executable by the one or more processors further to:
switch operation of the second and third coils between the first and second modes;
when in the first mode, manage operation of the electronic circuitry and the second and third coils to at least one of sense biological signals, deliver treatment for a non-physiologic condition or wirelessly communicate with at least one of an external device or second implanted device;
when in the second mode, manage operation of the electronic circuitry and the second and third coils to detect the second and third time varying gradients of the magnetic field along the second and third axes.

5. The IMD of claim 3, wherein the program instructions are executable by the one or more processors further to:
receive a manual input to switch from the first mode to the second mode; or
determine the system is within a static magnetic field and switch from the first mode to the second mode responsive to detection of the static magnetic field.

6. The IMD of claim 1, wherein the electronic circuitry includes a transceiver, and the first coil represents a telemetry coil, when in the first mode, the telemetry coil configured to wirelessly communicate with at least one of the external device or second implanted device.

7. The IMD of claim 1, wherein the first coil represents a transformer coil, when in the first mode, the transformer coil is configured to at least one of step-up or step-down a voltage between primary and secondary sides of the transformer to provide power at a select voltage to the electronic circuitry.

8. The IMD of claim 1, wherein the first coil represents a transformer coil coupled between a battery and a charge capacitor, when in the first mode, the transformer coil configured to step-up a voltage level from a battery to a select voltage level to charge a capacitor.

9. The IMD of claim 1, wherein the first coil is a telemetry coil, the second coil is a transformer coil, and the third coil is an auxiliary coil.

10. The IMD of claim 1, wherein the program instructions are executable by the one or more processors further to:
analyze the first time varying gradient of the magnetic field, the second time varying gradient of the magnetic field, and the third time varying MR generated gradient field to identify a signal generated by the magnetic field; and
discriminate between the signal generated by the magnetic field and the biological signals.

11. A method, comprising:
under control of one or more processors,
monitoring biological signals, or delivering a treatment for a non-physiologic condition, with a first coil, a second coil, and a third coil in a first mode;
detecting, with the first coil, a first time varying MR generated gradient field along a first axis in a second mode; and
discriminating between the biological signals and a signal generated by the magnetic field when diagnosing a medical condition based on the biological signals, or delivering the treatment.

12. The method of claim 11, further comprising:
under the control of the one or more processors,
detecting a static magnetic field; and
switching from the first mode to the second mode in response to detecting the static magnetic field.

13. The method of claim 11, further comprising: wherein the electronic circuitry includes a transceiver, and the first coil represents a transceiver coil, the transceiver coil, when in the first mode, configured to wirelessly communicate with at least one of the external device or second implanted device.

14. The method of claim 11, further comprising:
under the control of the one or more processors,
determining a period of time when the magnetic field is detectable; and
switching from the first mode to the second mode during the period of time.

15. The method of claim 11, wherein discriminating between the signal generated by the magnetic field and the biological signals comprises:
obtaining a second time varying MR generated gradient field along a second axis,
obtaining the third time varying MR generated gradient field along the third axis; and
determining the signal generated by the magnetic field based on the first time varying MR generated gradient field along the first axis, the second time varying MR generated gradient field along the second axis, and the third time varying MR generated gradient field along the third axis.

16. The method of claim 15, wherein discriminating between the biological signals and a signal generated by the magnetic field further comprises:
obtaining a candidate cardiac activity signal;
identifying the candidate cardiac activity signal as a cardiac activity signal based on the signal generated by the magnetic field.

17. The method of claim 16, wherein identifying the candidate cardiac activity signal as a cardiac activity signal comprises comparing the candidate cardiac activity signal to the signal generated by the magnetic field.

18. The method of claim 11, further comprising:
under the control of the one or more processors,
treating the non-physiologic condition in the second mode.

19. An implantable medical device (IMD), comprising:
a first coil configured to operate in first and second modes, when in the second mode, the first coil configured to detect a first time varying MR generated gradient field along a first axis;
a second coil configured to detect a second time varying MR generated gradient field along a second axis;
a third coil configured to detect a third time varying MR generated gradient field along a third axis;
electronic circuitry;
one or more processors; and
a memory coupled to the one or more processors, wherein the memory stores program instructions, wherein the program instructions are executable by the one or more processors to:
switch operation of the first coil between the first and second modes;
when in the first mode, manage operation of the electronic circuitry and the first coil to at least one of sense biological signals, deliver treatment for a non-physiologic condition or wirelessly communicate with at least one of an external device or second implanted device;
when in the second mode, manage operation of the electronic circuitry and the first coil to detect the first time varying MR generated gradient field along the first axis; and
and
determine when to switch from the first mode to the second mode.

20. The IMD of claim 19, wherein to determine when to switch from the first mode to the second mode, the program instructions are executable by the one or more processors further to:
detect a static magnetic field; and
switch from the first mode to the second mode in response to detecting the static magnetic field.
